(19) United States
(12) Patent Application Publication
(10) Pub. No.: US 2025/0143435 A1
(43) Pub. Date: May 8, 2025

(54) Tweezer Device

(71) Applicant: Nina D. Farzin, Potomac, MD (US)

(72) Inventor: Nina D. Farzin, Potomac, MD (US)

(21) Appl. No.: 18/940,122

(22) Filed: Nov. 7, 2024

Related U.S. Application Data
(60) Provisional application No. 63/547,664, filed on Nov. 7, 2023.

(51) Int. Cl. A45D 26/00; A61B 17/00; A61B 17/30

(52) U.S. Cl. CPC A45D 26/0066; A61B 2017/00057; A61B 2017/305

(57) ABSTRACT

A tweezer device is disclosed herein. The tweezer device includes a pair of angled tips, the pair of angled tips sized and configured to fit within a body cavity of a person or animal to remove a material or substance from the body cavity of the person or animal; and a tweezer body, the tweezer body having a first end that is distal from the pair of angled tips and a second end that is proximal to the pair of angled tips, the tweezer body including a shaft portion connected to a pair of angled connector portions, each of the angled connector portions being coupled to a respective one of the angled tips.

### CROSS-REFERENCE TO RELATED APPLICATIONS

This patent application claims priority to, and incorporates by reference in its entirety, U.S. Provisional Patent Application No. 63/547,664, entitled “Tweezer Device”, filed on Nov. 7, 2023.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### INCORPORATION BY REFERENCE OF MATERIAL SUBMITTED ON A COMPACT DISK

Not Applicable.

### BACKGROUND OF DISCLOSURE

### 1. Field of the Disclosure

The disclosure generally relates to a tweezer device. More particularly, the disclosure relates to a tweezer device that is configured to be used with a light assembly.

### 2. Background

Tweezers, a small hand tool, are used to grasp, grip, hold, or remove objects which may be too small for human fingers. There are various tweezer designs out in the market. The different types of tweezers include various tips such as slant tip, point tip, pointed slant tip, straight tip, arched claws, mini-tweezer, and wide grip tweezers. While all these are mostly used for grooming, these can be harmful in certain applications, such as where the tweezer tips are inserted into a child's nose and ear.

Therefore, what is needed is a tweezer device that is configured to be safely used in a variety of different applications, such as for removing a material or substance from a body cavity of a person or animal.

### SUMMARY OF THE DISCLOSURE

Accordingly, the present disclosure is directed to a tweezer device that substantially obviates one or more problems resulting from the limitations and deficiencies of the related art.

In accordance with one or more embodiments of the present disclosure, there is provided a tweezer device that includes a pair of angled tips, the pair of angled tips sized and configured to fit within a body cavity of a person or animal to remove a material or substance from the body cavity of the person or animal; and a tweezer body, the tweezer body having a first end that is distal from the pair of angled tips and a second end that is proximal to the pair of angled tips, the tweezer body including a tubular portion connected to a pair of angled connector portions, each of the angled connector portions being coupled to a respective one of the angled tips, and the tubular portion of the tweezer body defining a central passageway for receiving a shaft portion of a light assembly being used to illuminate an area of the body cavity of the person or animal from which the material or substance is being removed.

In a further embodiment of the present disclosure, the tweezer body further comprises a stop located proximal to the pair of angled tips, the stop sized to prevent insertion of the stop into the body cavity of the person or animal and limit the depth that the angled tips are able to be inserted into the body cavity of the person or animal.

In yet a further embodiment, the tweezer body further comprises a pair of animal head portions collectively forming the stop, each of the animal head portions being connected between a respective one of the angled connector portions and a respective one of the angled tips.

In still a further embodiment, the tweezer device further comprises the light assembly, the shaft portion of the light assembly configured to be removably received within the central passageway of the tubular portion of the tweezer body, the light assembly including a light emitting device, the light emitting device configured to emit light between the pair of angled connector portions so as to illuminate the area of the body cavity of the person or animal from which the material or substance is being removed.

In yet a further embodiment, the light assembly is attached to the tubular portion of the tweezer body, the light emitting device is disposed proximal to a location where the tubular portion of the tweezer body is connected to the pair of angled connector portions of the tweezer body.

In still a further embodiment, the light assembly further comprises a power source operatively coupled to the light emitting device, the power source configured to power the light emitting device, and the power source disposed inside a housing of the light assembly.

In yet a further embodiment, the light emitting device is in a form of a light-emitting diode.

In still a further embodiment, the tweezer device further comprises a base cap, the base cap including an outer end portion and an inner sleeve portion extending from the outer end portion, the inner sleeve portion of the base cap configured to be removably received within the central passageway of the tubular portion of the tweezer body such that the base cap forms a handle end cap on the first end of the tweezer body.

In yet a further embodiment, the tweezer device is formed from a polymeric material.

In still a further embodiment, the polymeric material forming the tweezer device does not comprise latex, bisphenol A, or polyvinyl chloride.

In accordance with one or more other embodiments of the present disclosure, there is provided a tweezer device that includes a pair of angled tips, the pair of angled tips sized and configured to fit within a body cavity of a person or animal to remove a material or substance from the body cavity of the person or animal; and a tweezer body, the tweezer body having a first end that is distal from the pair of angled tips and a second end that is proximal to the pair of angled tips, the tweezer body including a shaft portion connected to a pair of angled connector portions, each of the angled connector portions being coupled to a respective one of the angled tips.

In a further embodiment of the present disclosure, the tweezer body further comprises a stop located proximal to the pair of angled tips, the stop sized to prevent insertion of the stop into the body cavity of the person or animal and limit the depth that the angled tips are able to be inserted into the body cavity of the person or animal.

In yet a further embodiment, the tweezer body further comprises a pair of animal head portions collectively forming the stop, each of the animal head portions being connected between a respective one of the angled connector portions and a respective one of the angled tips.

In accordance with yet one or more other embodiments of the present disclosure, there is provided a tweezer device that includes a pair of angled tips, the pair of angled tips sized and configured to fit within a body cavity of a person or animal to remove a material or substance from the body cavity of the person or animal; a tweezer body, the tweezer body having a first end that is distal from the pair of angled tips and a second end that is proximal to the pair of angled tips, the tweezer body including a tubular portion connected to a pair of angled connector portions, each of the angled connector portions being coupled to a respective one of the angled tips, and the tubular portion of the tweezer body defining a central passageway; and a light assembly, the light assembly including a shaft portion configured to be removably received within the central passageway of the tubular portion of the tweezer body, the light assembly including a light emitting device, the light emitting device configured to emit light between the pair of angled connector portions so as to illuminate the area of the body cavity of the person or animal from which the material or substance is being removed.

In a further embodiment of the present disclosure, the light assembly is attached to the tubular portion of the tweezer body, the light emitting device is disposed proximal to a location where the tubular portion of the tweezer body is connected to the pair of angled connector portions of the tweezer body.

In yet a further embodiment, the tweezer device further comprises a base cap, the base cap including an outer end portion and an inner sleeve portion extending from the outer end portion, the inner sleeve portion of the base cap configured to be removably received within the central passageway of the tubular portion of the tweezer body such that the base cap forms a handle end cap on the first end of the tweezer body, the light assembly and the base cap configured to be interchangeably used with the tweezer body.

It is to be understood that the foregoing general description and the following detailed description are merely exemplary and explanatory in nature. As such, the foregoing general description and the following detailed description should not be construed to limit the scope of the appended claims in any sense.

### BRIEF DESCRIPTION OF THE SEVERAL VIEWS OF THE DRAWINGS

The device will now be described, by way of example, with reference to the accompanying drawings, in which:

FIG. 1 is a front-side perspective view of a tweezer device, according to an illustrative embodiment of the disclosure, wherein a base cap is provided on a distal end of the tweezer body;

FIG. 2 is a front elevational view of the tweezer device shown in FIG. 1;

FIG. 3 is a rear elevational view of the tweezer device shown in FIG. 1;

FIG. 4 is a first side view of the tweezer device shown in FIG. 1;

FIG. 5 is an opposite second side view of the tweezer device shown in FIG. 1;

FIG. 6 is a top plan view of the tweezer device the shown in FIG. 1;

FIG. 7 is a bottom plan view of the tweezer device shown in FIG. 1;

FIG. 8 is another front-side perspective view of the tweezer device shown in FIG. 1, wherein the base cap is shown removed from the tweezer body;

FIG. 9 is yet another front-side perspective view of the tweezer device shown in FIG. 1, wherein the tweezer body is provided with a light assembly inserted therein;

FIG. 10 is another front elevational view of the tweezer device shown in FIG. 1, wherein the light assembly is shown removed from the tweezer body; and

FIG. 11 is still another front-side perspective view of the tweezer device shown in FIG. 1, wherein the tweezer body is provided with the light assembly inserted therein, and a magnifying glass accessory is disposed on the tweezer device.

Throughout the figures, the same parts are always denoted using the same reference characters so that, as a general rule, they will only be described once.

### DETAILED DESCRIPTION OF ILLUSTRATED EMBODIMENT(S)

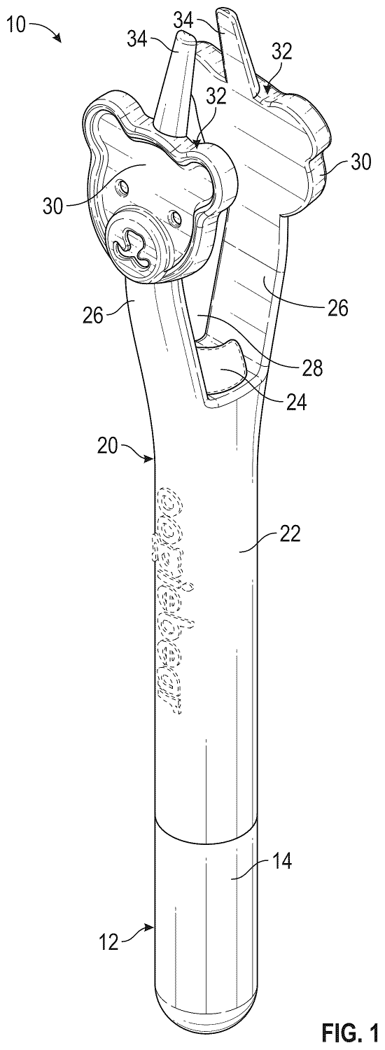

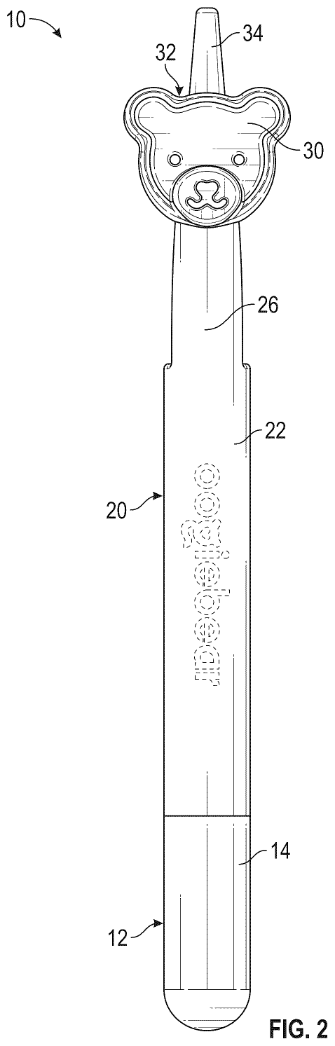

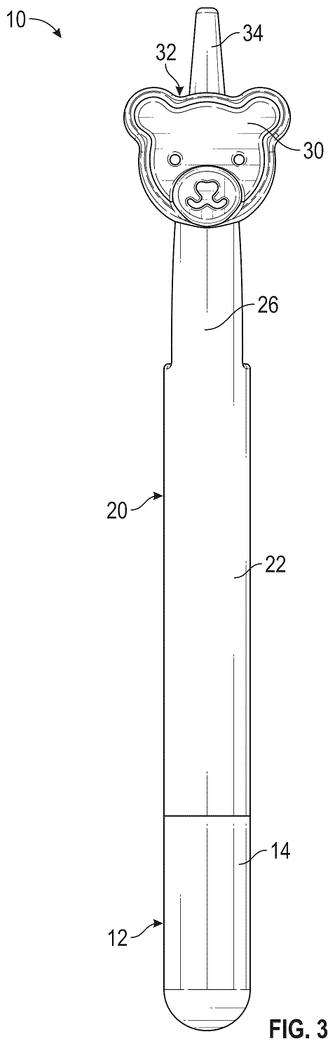

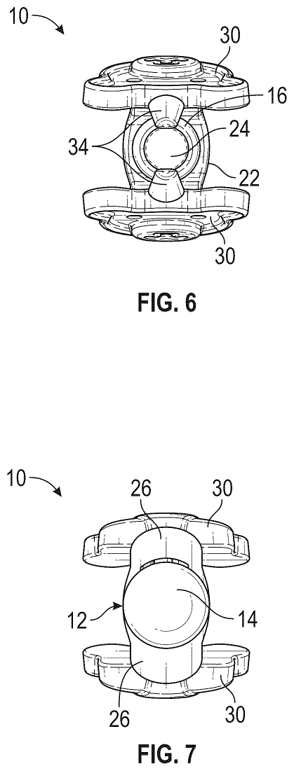

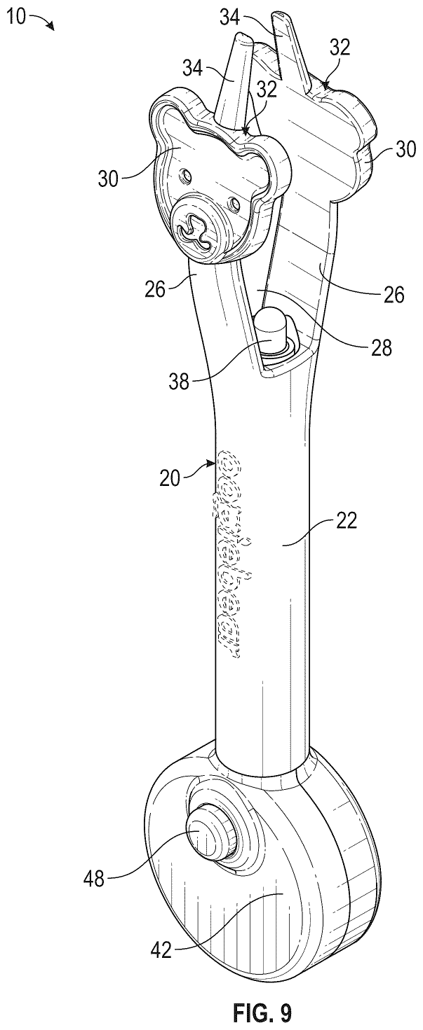

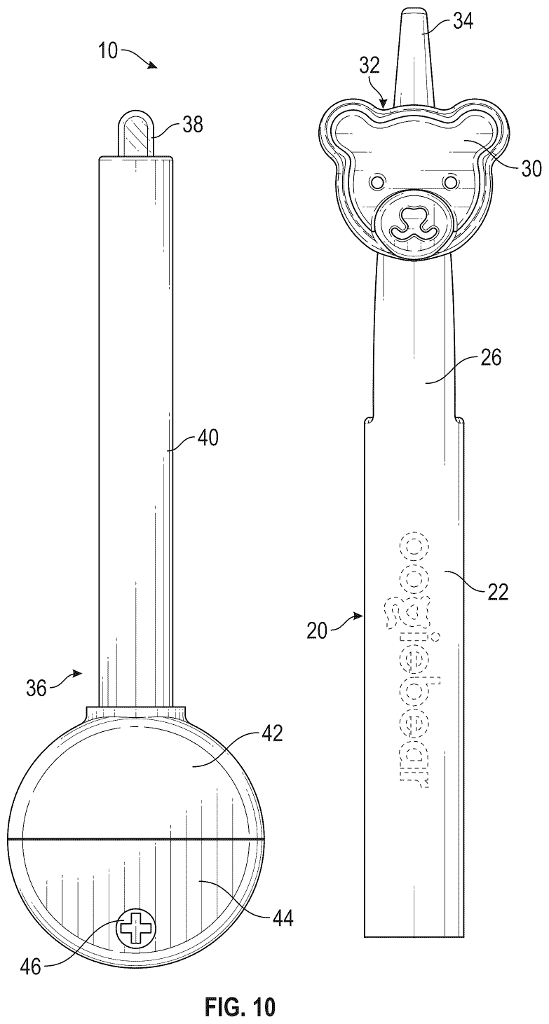

An illustrative embodiment of a tweezer device is seen generally at 10 in FIGS. 1-11. With initial reference to FIGS. 1 and 2, it can be seen that the tweezer device 10 includes a pair of angled tips 34, the pair of angled tips 34 sized and configured to fit within a body cavity of a person or animal to remove a material or substance from the body cavity of the person or animal; and a tweezer body 20, the tweezer body 20 having a first end that is distal from the pair of angled tips and a second end that is proximal to the pair of angled tips 34, the tweezer body 20 including a tubular portion 22 connected to a pair of angled connector portions 26, each of the angled connector portions 26 being coupled to a respective one of the angled tips 34, and the tubular portion 22 of the tweezer body 20 defining a central passageway 24 for receiving a shaft portion 40 of a light assembly 36 being used to illuminate an area of the body cavity of the person or animal from which the material or substance is being removed (see FIGS. 9 and 10).

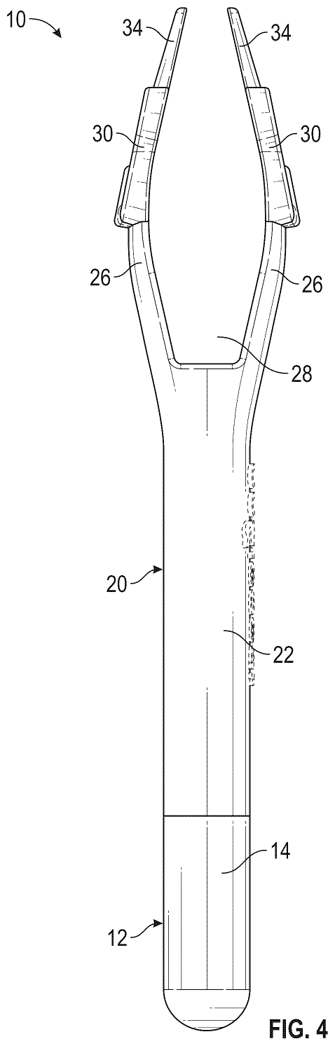

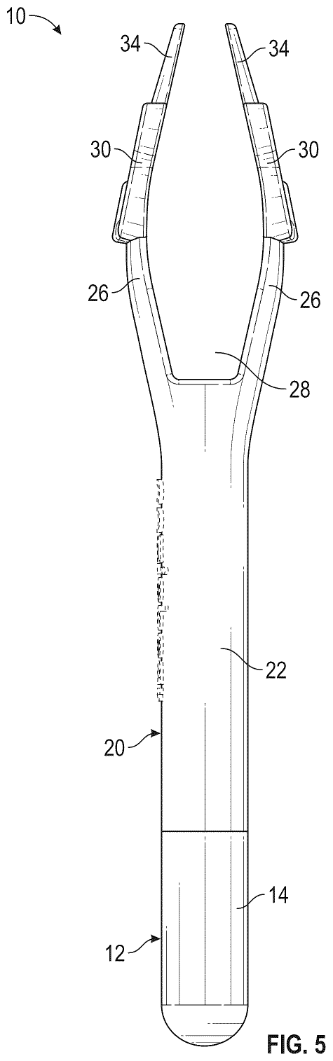

In the illustrative embodiment, as best shown in FIGS. 4 and 5, the angled connector portions 26 are angled outwardly from the tubular portion 22 of the tweezer body 20 at respective acute angles. Also, in the illustrative embodiment, the outward acute angles of the angled connector portions 26 are approximately equal or equal to one another. In FIGS. 4 and 5, it can be seen that a gap 28 is defined between the angled connector portions 26. Referring again to FIGS. 4 and 5, the angled tips 34 are angled inwardly toward one another at respective acute angles. In addition, in the illustrative embodiment, the inward acute angles of the angled tips 34 are approximately equal or equal to one another.

In the illustrative embodiment, referring to FIGS. 1-3, it can be seen that the tweezer body 20 further comprises a stop (e.g., formed by stop surfaces 32) located proximal to the pair of angled tips 34. The stop 32 is sized to prevent insertion of the stop 32 into the body cavity of the person or animal and limit the depth that the angled tips 34 are able to be inserted into the body cavity of the person or animal. Also, as shown in the illustrative embodiment of FIGS. 1-3, it can be seen that the tweezer body 20 further comprises a pair of animal head portions 30 collectively forming the stop 32. Each of the animal head portions 30 is connected between a respective one of the angled connector portions 26 and a respective one of the angled tips 34.

In the illustrative embodiment of the tweezer device 10, referring again to FIGS. 1-3, each of the head portions 30 is provided with ornamental features that resemble the head of a “teddy bear,” with the top of the heads forming stop surfaces 32. However, various other ornamental features may be used. For example, in other embodiments, the head portions 30 may take on other suitable shapes, such as but not limited to, the head shapes of other animals (e.g., other caricature animal head shapes). Alternatively, a flat stop surface without any ornamentation can be used.

In the illustrative embodiment, the tweezer device 10 is formed from a polymeric material that does not comprise latex, bisphenol A (BPA), or polyvinyl chloride (PVC) (i.e., the material is latex-free, BPA-free, and PVC-free). More specifically, in the illustrative embodiment, the polymeric material forming the tweezer device 10 comprises silicone that does not contain latex, BPA, or PVC.

In the illustrative embodiment, as best shown in FIGS. 9 and 10, it can be seen that the tweezer device 10 may further include a light assembly 36. The shaft portion 40 of the light assembly 36 is configured to be removably received within the central passageway 24 of the tubular portion 22 of the tweezer body 20. Also, referring to FIGS. 9 and 10, it can be seen that the light assembly 36 includes a light emitting device 38 at the one end thereof. The light emitting device 38 is configured to emit light between the pair of angled connector portions 26 so as to illuminate the area of the body cavity of the person or animal from which the material or substance is being removed. In FIG. 9, it can be seen that, when the light assembly 36 is attached to the tubular portion 22 of the tweezer body 20, the light emitting device 38 is disposed proximal to a location where the tubular portion 22 of the tweezer body 20 is connected to the pair of angled connector portions 26 of the tweezer body 20.

In the illustrative embodiment, the light assembly 36 further comprises a power source operatively coupled to the light emitting device 38. The power source is configured to power the light emitting device 38, and the power source is disposed inside a housing of the light assembly 36. In the illustrative embodiment, the light emitting device 38 is in a form of a light-emitting diode.

With combined reference to FIGS. 9 and 10, in the illustrative embodiment, the light assembly 36 generally comprises the shaft portion 40 that extends upwardly from a circular battery compartment end portion 42. The upper end of the shaft portion 40, which is opposite to the battery compartment end portion 42, comprises the light emitting device 38 with a dome-shaped light cover that is disposed over an internal light-emitting element. Also, as shown in the illustrative embodiment of FIG. 9, it can be seen that the front side of the battery compartment end portion 42 comprises a light power button 48 for activating and deactivating the light emitting device 38. Advantageously, the light power button 48 of the light assembly 36 is in a form of an easy-to-press on and off button 48 to help illuminate any small objects stuck in a person's skin or in a body cavity. The rear side of the battery compartment end portion 42 comprises a removable cover 44 for gaining access to the power source of the light assembly 36 (i.e., one or more batteries that provide power for the light emitting device 38—see FIG. 10). In the illustrative embodiment, as shown in FIG. 10, the removable cover 44 is removably secured to the housing of the battery compartment end portion 42 by a fastener 46 (e.g., screw 46). More specifically, in the illustrative embodiment, the battery compartment cover 44 is removably attached to the housing by two projections and the screw 46, which passes through a fastener aperture in the cover 44 and is threadingly received within a fastener boss of the housing section. In the illustrative embodiment, the electrical components, housing, and other features of the light assembly 36 may be as described in Applicant's pending U.S. patent application Ser. No. 17/412,185, the entire disclosure of which is incorporated herein by reference.

In the illustrative embodiment, the shaft portion 40 and the battery compartment end portion 42 forms a handle portion or grip portion of the tweezer device 10 that facilitates manipulation of the tweezer device 10 with the tweezer body 20 attached to the light assembly 36. As shown in the illustrative embodiment of FIG. 9, when the light assembly 36 is attached to the tubular portion 22 of the tweezer body 20, the tubular portion 22 of the tweezer body 20 has a second end at the light emitting device 38 of the light assembly 36 and a first end oppositely disposed relative to the second end. In the illustrative embodiment, the tubular portion 22 of the tweezer body 20 is configured to fit over the shaft portion 40 of the light assembly 36 and extend to the battery compartment end portion 42 of the light assembly 36. As shown in FIG. 9, the first end of the tubular portion 22 of the tweezer body 20 is exteriorly disposed proximate to the battery compartment end portion 42 of the light assembly 36.

In the illustrative embodiment, the shaft portion 40 of the light assembly 36 is an elongate member having a generally circular cross-section that is of length, width and thickness that comfortably facilitates gripping of the tweezer device 10 during use. The length of the shaft portion 40 can be from about 3 centimeters (cm) to about 10 cm, although a handle portion shorter than 3 cm or longer than 10 cm would also be expected to perform adequately.

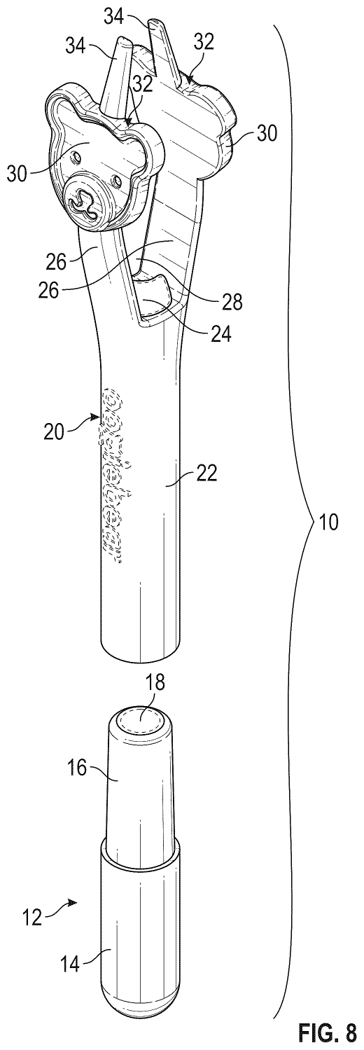

With particular reference to FIGS. 1 and 8, in the illustrative embodiment, the tweezer device 10 may further include a base cap 12. As shown in FIGS. 1 and 8, the base cap 12 includes an outer end portion 14 and an inner sleeve portion 16 extending from the outer end portion 14. The inner sleeve portion 16 of the base cap 12 is configured to be removably received within the central passageway 24 of the tubular portion 22 of the tweezer body 20 such that the base cap 12 forms a handle end cap on the first end of the tweezer body 20. In FIG. 8, it can be seen that the inner sleeve portion 16 of the base cap 12 has a central aperture 18 disposed therein.

In the illustrative embodiment, the tweezer body 20 is in a form of a tweezer attachment that is removably coupled to the light assembly 36 (see FIGS. 9 and 10). Other attachments, such as the first extraction portion and the second extraction portion described in U.S. patent application Ser. No. 17/412,185, may also be removably coupled to the light assembly 36. As such, in the illustrative embodiment, the tweezer body 20, the first extraction portion described in U.S. patent application Ser. No. 17/412,185, and the second extraction portion described in U.S. patent application Ser. No. 17/412,185 are configured to be interchangeably used with the light assembly 36 (i.e., a user can selectively attach either the tweezer body, the first extraction portion, or the second extraction portion to the light assembly 36 depending on the attachment portion that is desired). Also, in the illustrative embodiment, the base cap 12 and the light assembly 36 are configured to be interchangeably attached to the tweezer body 20 as part of a tweezer device kit.

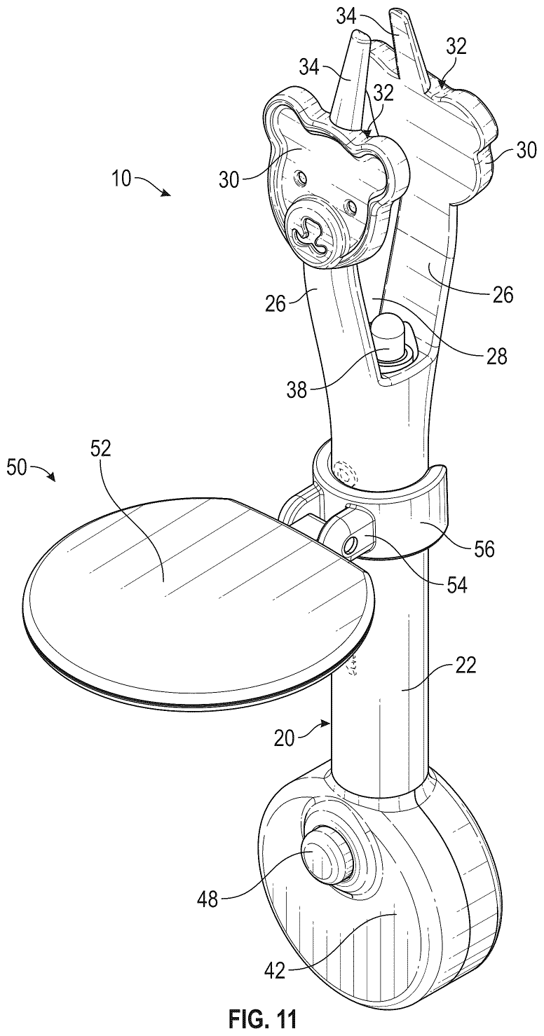

Now, with reference to FIG. 11, an illustrative embodiment of a magnifying glass accessory 50 that may be used with the tweezer device 10 will be explained. When the magnifying glass accessory 50 is clipped onto the tweezer device 10 (e.g., as shown in FIG. 11), the magnifying glass accessory 50 can be used for visually enlarging portions of the nostrils or ear canals of the infant or other person or animal on which the tweezer device 10 is being used. In the illustrative embodiment, as shown in FIG. 11, the magnifying glass accessory 50 is able to snap onto the tubular portion 22 of the tweezer body 20 so that a user is able to look through the magnifying lens of the magnifying glass accessory 50 to make small objects more easily visible. Initially, referring to FIG. 11, it can be seen that the magnifying glass accessory 50 generally comprises a magnifying disk portion 52, a hinge portion 54, and clip portion 56. In the illustrative embodiment, the magnifying disk portion 52 may comprise a convex lens that visually magnifies an object when a user looks at the object through the magnifying disk portion 52. The clip portion 56 of the magnifying glass accessory 50 may comprise a resilient clip that enables the magnifying glass accessory 50 to be removably clipped onto the tubular portion 22 of the tweezer body 20 of the tweezer device 10 (e.g., as shown in FIG. 11). Also, as shown in the illustrative embodiment of FIG. 11, the magnifying disk portion 52 is hingedly coupled to the clip portion 56 by means of the hinge portion 54 so that the magnifying disk portion 52 is able to rotate about the hinge axis so that different viewing angles of the user of the tweezer device 10 may be accommodated.

Now, with reference to FIGS. 1, 9, and 11, the manner in which the tweezer device 10 of the illustrative embodiment may be used will be described. In the illustrative embodiment, to use the tweezer device 10, a user may first slide the tubular portion 22 of the tweezer body 20 onto the shaft portion 40 of the light assembly 36, and then attach the magnifying glass accessory 50 to locate objects on or under the skin, or in a body cavity. The tweezer device 10 attached with the light assembly 36 is the perfect small hand tool, which allows a user to remove a material or substance from a body cavity of a person or animal. For example, a parent may extract boogers and earwax from his or her child safely and effectively without going too far into the nose and ear. It is readily apparent that the versatile tweezer device 10 has many other uses in addition to the extraction of boogers and earwax, such as being used for extracting hard-to-remove small objects, such as splinters and ticks. In one or more embodiments, parents may hold the tweezer device 10 with their fingers very close to the tip, which allows a more precise way of approaching their baby safely to enter the nostrils or ears. The light 38 allows the parent to insert the angled tips 34 of the tweezer device 10 into the nasal passage with a better view of the boogers. As shown in FIG. 11, the tweezer device 10 may also be provided with the magnifying glass accessory 50 to help get a better view of what is being grabbed by the angled tips 34 of the tweezer device 10. The tweezer device 10 is particularly well-suited for extracting boogers from the nostrils of babies and earwax from the ears of babies. Parents can also use a wipe to clean around the tips of the nostril.

It is readily apparent that the aforedescribed tweezer device 10 offers numerous advantages. As described above, the tweezer device 10 is configured to be safely used in a variety of different applications, such as for removing a material or substance from a body cavity of a person or animal. Also, in the illustrative embodiment, the tweezer device 10 is a lightweight tool which is safe to use, easy to carry, and easy to clean. The tubular portion 22 of the tweezer body 20 can slide on and off of the shaft portion 40 of the light assembly 36 for cleaning in between each use. Another advantageous feature of the tweezer device 10 is that the device 10 can be used manually with or without the light assembly 36 and/or magnifying glass accessory 50. The tweezer device 10 has an unlimited number of applications for humans plus pets. The tweezer device 10 may be used with or without the light base assembly 36.

In the illustrative embodiment, the tweezer device 10 advantageously incorporates various safety features. For example, as shown in the illustrative embodiment of FIG. 1, the ends of the angled tips 34 of the tweezer device 10 are rounded to enhance the safety of the device 10. Also, the tweezer device 10 has a stopping mechanism 32 to create a safe working length to prevent over-insertion of the angled tips 34. For example, when used with a baby, the stopping mechanism 32 advantageously prevents over-insertion that could potentially hurt the baby's nose/face or ears. The stopping mechanism 32 formed by the pair of animal head portions 30 ensures that the angled tips 34 are not inserted too deep. The angled tips 34 of the tweezer device 10 facilitate the removal of boogers and ear wax, but sometimes it may become difficult to see what is trying to be removed. As such, the light 38 of the light assembly 36 may also be very helpful. Along with light 38, the rigidity of the tweezer device 10 allows for purposeful closure without accidentally pinching the person or animal on which the device 10 is being used.

In the illustrative embodiment, the thermoplastic tweezer device 10 may include a light assembly 36 and magnifying glass accessory 50, which is designed to remove a material or substance from a body cavity of a person or animal. For example, the tweezer device 10 may be used to remove boogers and earwax from the noses and ears of babies 0+ months in age. The tweezer device 10 may also be used to remove ticks from the ears of animals, or splinters from kids, etc. The tweezer device 10 utilizes the pair of animal head portions 30 as a protective stopping mechanism 32, ensuring the tweezer device 10 does not go too far into the body cavity of the person or animal on which it is being used. As such, the animal head portions 30 of the tweezer device 10 are both functional and visually attractive. The tweezer device 10 may also include the plastic magnifying glass accessory 50 described above to help get a better view of what is being removed from the body cavity of the person or animal (e.g., what is being picked out of the nose and ears of a baby). Along with a magnifier glass accessory 50, the tweezer device 10 may further include the base cap 12, which is interchangeable with a light base assembly 36. The light 38 of the light base assembly 36 helps get a better view of what is being removed from the body cavity of the person or animal.

Any of the features or attributes of the above described embodiments and variations can be used in combination with any of the other features and attributes of the above described embodiments and variations as desired.

Although the tweezer device 10 has been shown and described with respect to a certain embodiment or embodiments, it is apparent that the aforedescribed tweezer device 10 can be embodied in many different forms and that many other modifications and variations are possible without departing from the spirit and scope of the claimed invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A tweezer device, comprising:
a pair of angled tips, the pair of angled tips sized and configured to fit within a body cavity of a person or animal to remove a material or substance from the body cavity of the person or animal; and
a tweezer body, the tweezer body having a first end that is distal from the pair of angled tips and a second end that is proximal to the pair of angled tips, the tweezer body including a tubular portion connected to a pair of angled connector portions, each of the angled connector portions being coupled to a respective one of the angled tips, and the tubular portion of the tweezer body defining a central passageway for receiving a shaft portion of a light assembly being used to illuminate an area of the body cavity of the person or animal from which the material or substance is being removed.

2. The tweezer device according to claim 1, wherein the tweezer body further comprises a stop located proximal to the pair of angled tips, the stop sized to prevent insertion of the stop into the body cavity of the person or animal and limit the depth that the angled tips are able to be inserted into the body cavity of the person or animal.

3. The tweezer device according to claim 2, wherein the tweezer body further comprises a pair of animal head portions collectively forming the stop, each of the animal head portions being connected between a respective one of the angled connector portions and a respective one of the angled tips.

4. The tweezer device according to claim 1, further comprising:
the light assembly, the shaft portion of the light assembly configured to be removably received within the central passageway of the tubular portion of the tweezer body, the light assembly including a light emitting device, the light emitting device configured to emit light between the pair of angled connector portions so as to illuminate the area of the body cavity of the person or animal from which the material or substance is being removed.

5. The tweezer device according to claim 4, wherein, when the light assembly is attached to the tubular portion of the tweezer body, the light emitting device is disposed proximal to a location where the tubular portion of the tweezer body is connected to the pair of angled connector portions of the tweezer body.

6. The tweezer device according to claim 4, wherein the light assembly further comprises a power source operatively coupled to the light emitting device, the power source configured to power the light emitting device, and the power source disposed inside a housing of the light assembly.

7. The tweezer device according to claim 4, wherein the light emitting device is in a form of a light-emitting diode.

8. The tweezer device according to claim 1, further comprising:
a base cap, the base cap including an outer end portion and an inner sleeve portion extending from the outer end portion, the inner sleeve portion of the base cap configured to be removably received within the central passageway of the tubular portion of the tweezer body such that the base cap forms a handle end cap on the first end of the tweezer body.

9. The tweezer device according to claim 1, wherein the tweezer device is formed from a polymeric material.

10. The tweezer device according to claim 9, wherein the polymeric material forming the tweezer device does not comprise latex, bisphenol A, or polyvinyl chloride.

11. A tweezer device, comprising:
a pair of angled tips, the pair of angled tips sized and configured to fit within a body cavity of a person or animal to remove a material or substance from the body cavity of the person or animal; and
a tweezer body, the tweezer body having a first end that is distal from the pair of angled tips and a second end that is proximal to the pair of angled tips, the tweezer body including a shaft portion connected to a pair of angled connector portions, each of the angled connector portions being coupled to a respective one of the angled tips.

12. The tweezer device according to claim 11, wherein the tweezer body further comprises a stop located proximal to the pair of angled tips, the stop sized to prevent insertion of the stop into the body cavity of the person or animal and limit the depth that the angled tips are able to be inserted into the body cavity of the person or animal.

13. The tweezer device according to claim 12, wherein the tweezer body further comprises a pair of animal head portions collectively forming the stop, each of the animal head portions being connected between a respective one of the angled connector portions and a respective one of the angled tips.

14. A tweezer device, comprising:
a pair of angled tips, the pair of angled tips sized and configured to fit within a body cavity of a person or animal to remove a material or substance from the body cavity of the person or animal;
a tweezer body, the tweezer body having a first end that is distal from the pair of angled tips and a second end that is proximal to the pair of angled tips, the tweezer body including a tubular portion connected to a pair of angled connector portions, each of the angled connector portions being coupled to a respective one of the angled tips, and the tubular portion of the tweezer body defining a central passageway; and
a light assembly, the light assembly including a shaft portion configured to be removably received within the central passageway of the tubular portion of the tweezer body, the light assembly including a light emitting device, the light emitting device configured to emit light between the pair of angled connector portions so as to illuminate the area of the body cavity of the person or animal from which the material or substance is being removed.

15. The tweezer device according to claim 14, wherein, when the light assembly is attached to the tubular portion of the tweezer body, the light emitting device is disposed proximal to a location where the tubular portion of the tweezer body is connected to the pair of angled connector portions of the tweezer body.

16. The tweezer device according to claim 14, further comprising:
a base cap, the base cap including an outer end portion and an inner sleeve portion extending from the outer end portion, the inner sleeve portion of the base cap configured to be removably received within the central passageway of the tubular portion of the tweezer body such that the base cap forms a handle end cap on the first end of the tweezer body, the light assembly and the base cap configured to be interchangeably used with the tweezer body.
